SEQUENCE LISTING (2 sequences)

SEQ ID NO: 1 - protein, 223 aa, Streptomyces peucetius
  SpOMT2884 protein
MTQDQWQAVDAYISDLLVPEDDALAEALTASDAAGLPRINVAPNQGKLLHLLARVQGARTALEIGTLGGYSTIWLARALPADGRLISLEYDPAHAEVARANIARAGLDKVVEVRTGAALDTLPRIAAEPGAGPFDLVFIDADKRNNARYVEWALRLTRPGSVIIVDNVVRGGAVVDGSSEDPSIVGTREMFELVAREARLEATAVQTVGVKGYDGMLVARVIA

SEQ ID NO: 2 - DNA, 681 bp, Artificial Sequence
  NdeI-SpOMT2884-XhoI
catatgactcaagatcaatggcaagctgttgatgcctacatttctgacttgttggttccagaagatgatgcattggctgaagctttgactgcttctgatgctgctggtttgccaagaattaacgttgctccaaatcagggaaagttgttgcatttgttggctagagttcaaggtgctagaactgccttggaaattggtacattgggtggttactctactatttggttggccagagcattgccagctgatggaagattgatttctttggagtacgatccagcccatgctgaagttgctagagctaacattgcaagagccggattggataaggttgttgaagttagaacaggtgcagctttggacactttgcctagaatcgccgctgaaccaggtgctggtccttttgacttggtcttcatcgacgccgataagagaaacaacgctagatacgttgaatgggctttgagattgactagaccaggttctgttattatcgttgataacgtcgttagaggtggtgctgttgtcgacggttcttctgaagatccttctattgtcggtactagagagatgtttgaattggttgccagagaagcaagattggaggctaccgccgttcaaactgttggtgtcaagggttacgatggtatgttggtcgcaagagtcattgcctaactcgag

(19) United States
(12) Patent Application Publication
(10) Pub. No.: US 2017/0198318 A1
(43) Pub. Date: Jul. 13, 2017

(54) METHOD OF MANUFACTURING METHOXY-ISOFLAVONES BY BIOTRANSFORMATION AND USE THEREOF

(71) Applicants: NATIONAL UNIVERSITY OF TAINAN, Tainan City (TW); BIO-JOURDENESS INTERNATIONAL GROUP CO., LTD., Taichung City (TW)

(72) Inventors: TE-SHENG CHANG, TAINAN CITY (TW); YA-TING TSAI, TAINAN CITY (TW); HSIOU-YU DING, TAINAN CITY (TW); CHIEN-MIN CHIANG, TAINAN CITY (TW)

(21) Appl. No.: 15/389,849

(22) Filed: Dec. 23, 2016

(30) Foreign Application Priority Data
Jan. 13, 2016 (TW) 105100958

(51) Int. Cl. C12P 17/06; A61K 8/49; A61Q 19/02

(52) U.S. Cl. CPC C12P 17/06; C12Y 201/01; A61Q 19/02; A61K 8/498

(57) ABSTRACT

A method of manufacturing methoxy-isoflavones by biotransformation and a use thereof are revealed herein. The method comprises the steps of synthesizing a nucleic acid sequence including a SpOMT2884 gene ofStreptomyces peucetius; cloning the nucleic acid sequence into an expression vector to form a cyclic recombinant plasmid; transforming the cyclic recombinant plasmid into a microbial expression system (Escherichia coli); and incubating the microbial expression system in a medium containing 8-hydroxydaidzeins therein for generating methoxy-isoflavones.

### BACKGROUND OF THE INVENTION

Field of the Invention

The present invention relates to a method of manufacturing methoxy-isoflavones by biotransformation. It mainly comprises constructing a microbial host carrying a cyclic recombinant plasmid harboring a SpMT2884 gene sequence of Streptomyces peucetius and further incubating the microbial host in a medium containing 8-hydroxydaidzeins therein for a period of time to generate methoxy-isoflavones having activity of inhibiting melanogenesis.

Descriptions of Related Art

Isoflavone usually exists in some plants, such as soybeans, so it is also known as soy isoflavones. Two major isoflavones found in soybeans are daidzein and genistein, which have been demonstrated to prevent hormone-dependent diseases. In recent years, more studies have focused on isoflavones due to their pharmaceutical activities, including anti-cellular proliferation, aldose reductase inhibition, tyrosinase inhibition, anti-mutagenesis, anti-melanogenesis, enhancement of cancer chemotherapeutic activity, and the like. After a microbial fermentation, isoflavone derivatives, e.g. methyl-isoflavones, possess superior stability, bioactivity, cell membrane permeability and bioavailability to that of non-methylated isoflavones. For example, 4′-methoxydaidzein (also known as formononetin) was shown to have 10-fold anti-melanogenesis activity compared to that of daidzein.

In biological systems, O-methyltransferase (OMT) acts as an important enzyme for catalyzing the production of methoxy-isoflavone by substituting a methyl group for a hydrogen atom (H) of the hydroxyl group (—OH group) to form a methoxy group (—OCH₃ group). OMTs are ubiquitous in nature. Many OMTs have been found in various plants or microorganisms. Most plant OMTs possess high substrate specificity. In contrast, OMTs from microorganisms contain more flexibility in substrate specificity than those from plants. Some studies reported that O-methylation of isoflavones would increase biological activities of the isoflavones.

Due to the rarity of methoxy-isoflavones in nature, people recently focused on using biotransformation method for mass producing methoxy-isoflavones. For instance, U.S. Pat. No. 7,432,425 B2, issued on 7 Oct. 2008, disclosed an isoflavonoid methylation enzyme. It describes a method of cloning a 7-O-methyltransferase (7-OMT) gene derived from plants into alfalfa, so as to make the transgenic plant converts daidzein to 4′-O-methlated-isoflavonoid.

An OMT from Streptomyces peucetins ATCC 27952 (SpOMT2884) was reported to catalyze O-methylation of 7, 8-dihydroxyflavone to produce 7-hydroxy-8-methoxyflavone with antioxidant activity (Journal of Biotechnology, 2014, volume 184, page 128-137). Although many OMTs have been studied, whether the OMTs have 8-O-methylation activity toward isoflavones hasn't been confirmed yet.

At present, there are various kinds of flavonoids or isoflavones existed with specific characteristics. In order to increase the widespread use of soy isoflavones in cosmetic, healthcare or pharmaceutical compositions, it is worthy to convert the isoflavones to different kinds of methoxy-isoflavones by biotransformation and to explore special bioactivities of methoxy-isoflavones.

### SUMMARY OF THE INVENTION

The primary object of the present invention is to provide a method of manufacturing methoxy-isoflavones by biotransformation, including using an O-Methyltransferase SpOMT2884 from Streptomyces peucetius to convert 8-hydroxydaidzeins to methoxylated 8-hydroxydaidzeins (a.k.a methoxy-isoflavones) having activity of inhibiting melanogenesis.

Disclosed herein is a method of manufacturing methoxy-isoflavones by biotransformation comprising the steps of: (a) synthesizing a nucleic acid sequence having a SpOMT2884 gene of Streptomyces peucetius, a first restriction enzyme cleavage site and a second restriction enzyme cleavage site, wherein the first and the second restriction enzyme cleavage sites are respectively located upstream and downstream of the SpOMT2884 gene of Streptomyces peucetius; (b) cleaving the nucleic acid sequence with the first restriction enzyme and the second restriction enzyme; (c) cleaving an expression vector with the first restriction enzyme and the second restriction enzyme, and cloning the nucleic acid sequence into the expression vector to form a cyclic recombinant plasmid; and (d) transforming the cyclic recombinant plasmid into a microbial expression system and incubating the microbial expression system in a medium containing 8-hydroxydaidzeins therein for generating methoxy-isoflavones.

According to an embodiment of the present invention, the SpOMT2884 gene of Streptomyces peucetius encodes an O-methyltransferase and comprises an amino acid sequence of SEQ ID NO:1.

According to an embodiment of the present invention, the first restriction enzyme is NdeI and the second restriction enzyme is XhoI. Preferentially, the expression vector is pETDuet™ and the microbial expression system is Escherichia coli.

According to an embodiment of the present invention, the methoxy-isoflavones are 7,4′-dihydroxy-8-methoxy-isoflavone and 8,4′-dihydroxy-7-methoxy-isoflavone, respectively represented by the following structural formula I and formula II:

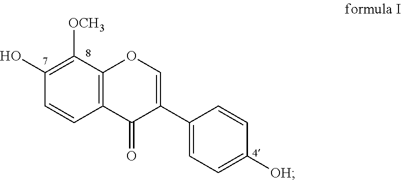 [SMILES: COc1c(O)ccc2c(=O)c(-c3ccc(O)cc3)coc12]

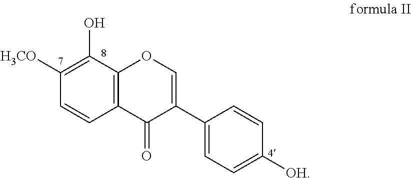 [SMILES: COc1ccc2c(=O)c(-c3ccc(O)cc3)coc2c1O]

According to an embodiment of the present invention, the methoxy-isoflavones are used in cosmetic composition for whitening. An effective dose of methoxy-isoflavone is applied to a subject in need for inhibiting melanin synthesis. The effective dose is preferentially ranging from 6.25 μM to 25 μM.

Thereby methoxy-isoflavones according to the present invention are further used in cosmetic composite for skin whitening such as water base cosmetics, emulsion cosmetics, ointment base cosmetics, power cosmetics and the like. Methoxy-isoflavone can also be added into other functional cosmetics such as humectants, or anti-wrinkle cosmetics.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 shows a flow diagram of manufacturing methoxy-isoflavones by biotransformation according to the present invention;

FIG. 2 shows a schematic diagram of a cyclic recombinant plasmid according to the present invention;

FIG. 3 shows an electrophoretogram of SpOMT2884 protein;

FIG. 4 shows UPLC profiles of fermentation broths containing 8-hydroxydaidzein standard, 0 h induction or 24 h isopropyl-β-D-thiogalactopyranoside (IPTG) induction of recombinant E. coli expressing SpOMT2884;

FIG. 5 shows a schematic diagram of a biotransformation of methoxy-isoflavones from 8-hydroxydaidzeins according to the present invention;

FIG. 6 shows production yields of methoxy-isoflavones according to the present invention;

FIG. 7 shows cytotoxicity of methoxy-isoflavones and effect of methoxy-isoflavones under different treatment conditions on melanogenesis according to the present invention.

### DETAILED DESCRIPTION OF THE PREFERRED EMBODIMENT

A method of manufacturing methoxy-isoflavones by biotransformation is disclosed herein, comprising the steps of:

(a) synthesizing a nucleic acid sequence having a SpOMT2884 gene of Streptomyces peucetius, a first restriction enzyme cleavage site and a second restriction enzyme cleavage site, wherein the first restriction enzyme cleavage site and the second restriction enzyme cleavage site are respectively located upstream and downstream of the SpOMT2884 gene of Streptomyces peucetius;

(b) cleaving the nucleic acid sequence with the first restriction enzyme and the second restriction enzyme, such as NdeI and XhoI, respectively;

(c) cleaving an expression vector (preferably an expression vector of pETDuet™) with the first restriction enzyme and the second restriction enzyme, and cloning the nucleic acid sequence into the expression vector to form a cyclic recombinant plasmid; and

(d) transforming the cyclic recombinant plasmid into a microbial expression system and incubating the microbial expression system (preferably Escherichia coli) in a medium containing 8-hydroxydaidzeins therein for generating methoxy-isoflavones.

The SpOMT2884 gene of Streptomyces peucetius encodes an O-methyltransferase and may, for example, comprises or be an amino acid sequence of SEQ ID NO:1. Preferably, the methoxy-isoflavone are 7,4′-dihydroxy-8-methoxy-isoflavone and 8,4′-dihydroxy-7-methoxy-isoflavone.

A use of the methoxy-isoflavones in cosmetic composition for skin whitening is also disclosed, comprising applying an effective dose of methoxy-isoflavones to a subject in need for inhibiting melanin synthesis. The effective dose is preferentially ranging from 6.25 μM to 25 μM, and the methoxy-isoflavone are 7,4′-dihydroxy-8-methoxy-isoflavone and 8,4′-dihydroxy-7-methoxy-isoflavone.

Hereinafter, an exemplary embodiment of the present invention will be described in detail with reference to the accompanying drawings.

Briefly, the present invention sets up a condition for biotransforming methoxy-isoflavones from 8-hydroxydaidzeins and mass production of methoxy-isoflavones in a fermentation tank, and further finds that the methoxy-isoflavones have activity of anti-melanin production.

### Experiment 1: Constructing a Cyclic Recombinant Plasmid Harboring a SpOMT2884 Gene of Streptomyces peucetius

In an embodiment of the present invention, methoxy-isoflavones are prepared by the following biotransformation.

### (1) Microorganisms and Materials

A. oryzae BCRC 32288 was purchased from the Bioresources Collection and Research Center (BCRC, Food Industry Research and Development Institute, Hsinchu, Taiwan). The expression system containing both E. coli BL21 (DE3) and vector pETDuet-1™ was obtained from Novagen. Isopropyl-β-D-1-thiogalactopyranoside (IPTG), 3-(4,5-dimethylthiazol-2-yl)-2, 5-diphenyltetrazolium bromide (MTT), and 3-isobutyl-1-methylxanthine (IBMX) were purchased from Sigma (St. Louis, Mo., USA). The 8-Hydroxydaidzein was isolated from 10 L fermentation broth of A. oryzae BCRC 32288 with 150 mg/L of daidzein feeding in yeast peptone dextrose medium according to the previous report [Biosci. Biotechnol. Biochem. 2007; 71:1330-1333]. The other reagents and solvents used were of high-quality and were purchased from commercially available sources.

(2) Expression of SpOMT2884 in Recombinant E. coli

The SpOMT2884 gene of Streptomyces peucetius was cloned into a pETDuet-1™ vector regulated by IPTG for investigating the biotransformation of methoxy-isoflavones from 8-hydroxydaidzeins.

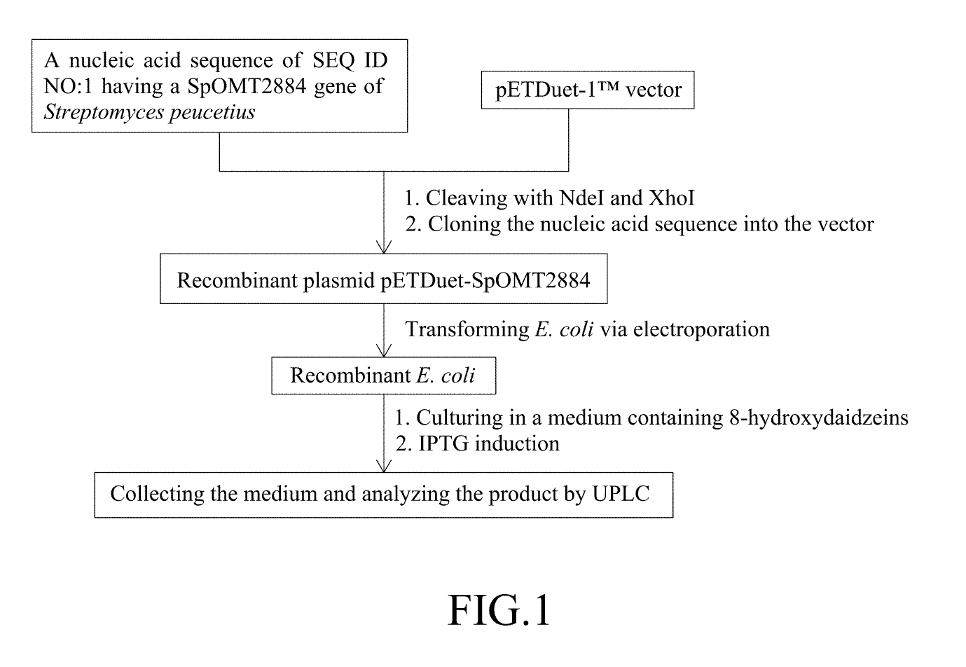

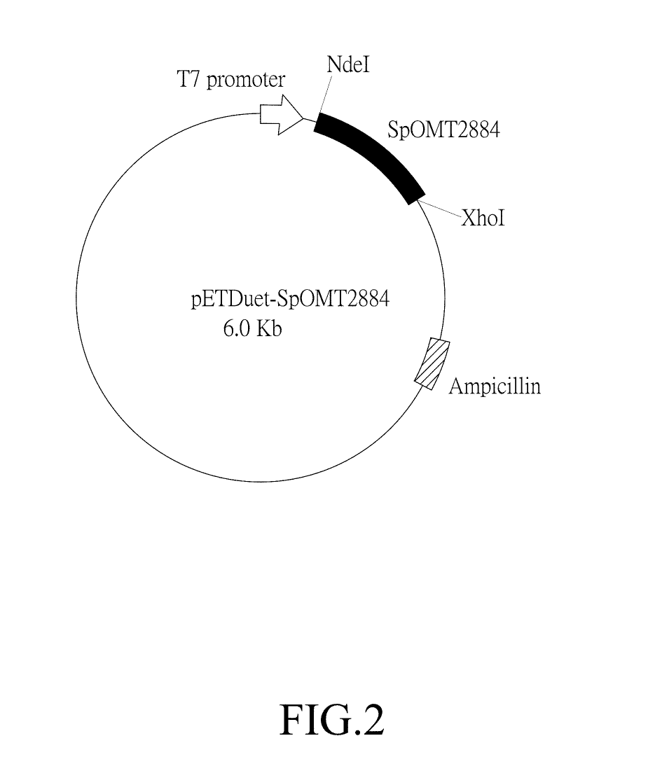

Referring to FIG. 1 and FIG. 2, a flow diagram of manufacturing methoxy-isoflavones by biotransformation and a schematic diagram of a cyclic recombinant plasmid according to the present invention are disclosed. A nucleic acid sequence of SEQ ID NO:1 (681 bp) having a Streptomyces SpOMT2884 gene (GenBank protein database accession number KF420279), a NdeI cleavage site and a XhoI cleavage site is synthesized with codon optimization based on the preferences of E. coli by GenScript (Piscataway, N.J., USA), which encodes an O-methyltransferase and comprises an amino acid sequence of SEQ ID NO: 1. The synthesized SpOMT2884 gene was subcloned into the pETDuet-1™ vector through the NdeI and XhoI sites to obtain the cyclic recombinant plasmid pETDuet-SpOMT2884. The expression vector was transformed into E. coli BL21 (DE3) via electroporation to obtain the recombinant E. coli used for the biotransformation. Since the optimal culture conditions and high replication rate of E. coli are widely known, and E. coli can be induced to considerably express target products such as induced by IPTG. Therefore, the present invention adopts E. coli as a microbial expression system herein.

The recombinant E. coli harboring expression vector was cultivated in 20 mL of LR medium (LeMaster and Richards minimal medium) containing 50 μg/mL of ampicillin and 0.4% glycerol, with 200 rpm shaking at 37° C. As the optical density at 600 nm reached 0.6, 0.5 mM of IPTG and 0.1 mM of 8-hydroxydaidzein were added to induce expression of SpOMT2884 and start the biotransformation. After a 4 h IPTG induction or an 8 h IPTG induction, the E. coli protein was collected and sodium dodecyl sulfate polyacrylamide gel electrophoresis (SDS-PAGE) was performed to confirm the production of SpOMT2884 protein. The technique to collect E. coli protein and perform SDS-PAGE are well-known to the people skilled in the art.

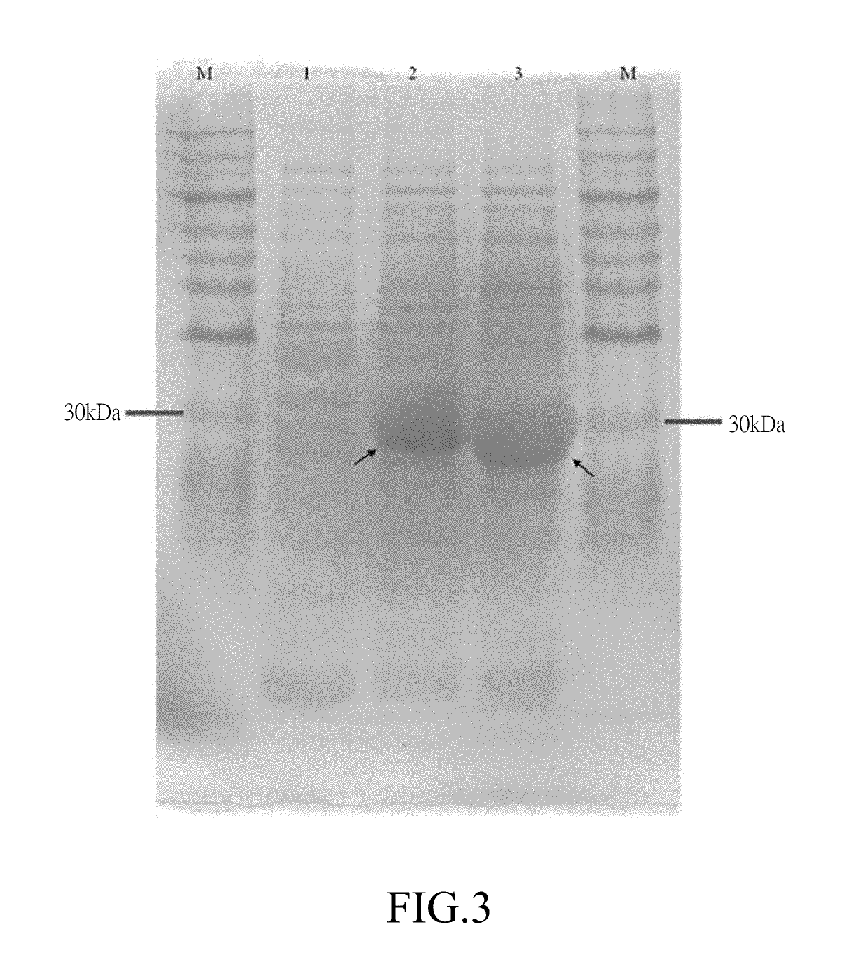

Referring to FIG. 3, an electrophoretogram of SpOMT2884 protein is disclosed. Crude proteins from whole cell lysis with 0 h IPTG induction (lane 1), 4 h IPTG induction (lane 2), and 8 h IPTG induction (lane 3) were separated with SDS-PAGE. Arrows indicate the expressed SpOMT2884 with about 30 kilodaltons (kDa). M represents the molecular weight markers. Compared with lane 1, the recombinant E. coli harboring the expression vector (lane 2 and lane 3) were confirmed to produce considerable SpOMT2884 protein after IPTG induction, and the expression of SpOMT2884 protein was increased with the increase of IPTG induction time.

### Experiment 2: Fermentation and Ultra Performance Liquid Chromatography (UPLC)

The recombinant E. coli harboring expression vector was cultivated in 20 mL of LR medium (LeMaster and Richards minimal medium) containing 50 μg/mL of ampicillin and 0.4% glycerol, with 200 rpm shaking at 37° C. As the optical density at 600 nm reached 0.6, 0.5 mM of IPTG and 0.1 mM of 8-hydroxydaidzein were added to induce expression of SpOMT2884 and start the biotransformation. At indicated time intervals, 0.5 mL of cultures was collected and analyzed with UPLC to detect the content of methoxy-isoflavones.

For the scale-up fermentation, a seed culture of recombinant E. coli (100 mL) was inoculated into a 5 L fermenter containing 2.5 L LR medium supplemented with 0.4% glycerol, followed by cultivation with aeration (0.5 vvm) and agitation (280 rpm) at 37° C. As the optical density at 600 nm reached 0.6, 0.5 mM of IPTG and 0.1 mM of 8-hydroxydaidzein were added to induce expression of SpOMT2884 and continue the fermentation for another 24 h. A 10 mL aliquot of the culture was collected at several different time intervals and analyzed with UPLC to check whether the substrate could be converted by the recombinant cells.

The purification process was the same as in the previous work [Process Biochem. 2015; 50:1614-1617] and is described briefly below. Two batches of 2.5 L fermentation broth were prepared for the purification of the biotransformation products. Following the fermentation, total broth was twice extracted with an equal volume of ethyl acetate, and the extracts were combined and condensed under a vacuum. The residue was then suspended in 200 mL of 50% methanol. After filtration with a 2.2 μm nylon membrane under a vacuum, the metabolite was purified by preparative high-performance liquid chromatography (HPLC) methods.

The operational conditions for the preparative HPLC analysis by a preparative C18 reversed-phase column (Inertsil, 10 μm, 20.0 i.d.×250 mm, ODS 3, GL Sciences, Eindhoven, The Netherlands) included a gradient elution using water (A) containing 1% (v/v) acetic acid and methanol (B) with a linear gradient for 25 min with 25% to 50% methanol (B) at a flow rate of 15 mL/min, and detection of the absorbance at 260 nm. The injection volume was 10 mL. The elution corresponding to the peak of the metabolite in the UPLC analysis was collected, condensed under a vacuum, and then crystallized by freeze-drying. Finally, 17.5 mg of compound (1) and 16.0 mg of compound (2) were obtained, and the structures of the compounds were confirmed with nuclear magnetic resonance (NMR) and mass spectral analysis.

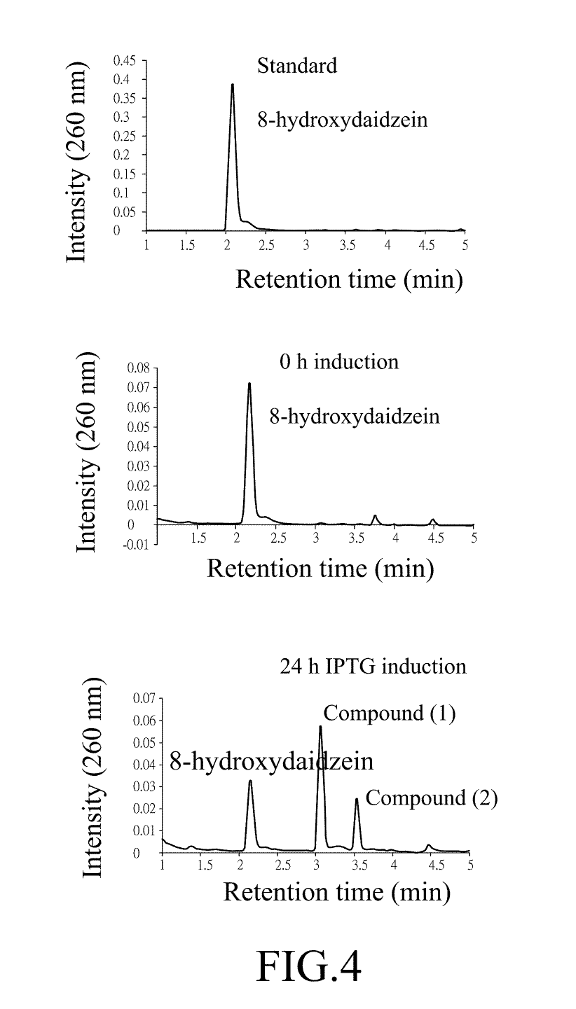

Referring to FIG. 4, UPLC profiles of fermentation broths containing 8-hydroxydaidzein standard, 0 h induction or 24 h isopropyl-β-D-thiogalactopyranoside (IPTG) induction of recombinant E. coli expressing SpOMT2884 are revealed. The results are shown while the Y axis represents absorbance intensity measured at 260 nm and the X axis represents the retention time. The 8-hydroxydaidzein standard appeared at retention times of 2.1 min. Two major metabolites, compounds (1) and (2), appeared as new peaks at retention times of 3.2 and 3.7 min, respectively, in the profile of the fermentation broth at 24 h of incubation.

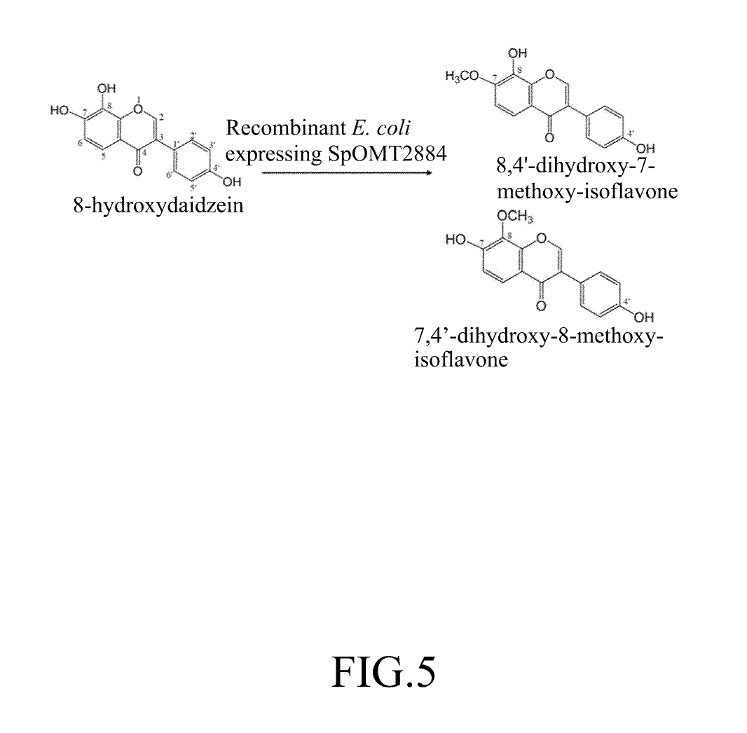

The metabolites were further isolated using a preparative HPLC method and were identified using spectrophotometric methods. Compound (1) showed an [M-H]⁺ ion peak at m/z: 283 in the electrospray ionization mass (ESI-MS) spectrum corresponding to the molecular formula C₁₆H₁₂O₅. Then ¹H and ¹³C NMR was performed to elucidate the structure. The full assignment of the ¹H and ¹³C NMR signals was conducted according to heteronuclear multiple quantum coherence (HMQC), heteronuclear multiple bond connectivity (HMBC), and correlation spectroscopy (COSY) spectra. The following data were collected for compound (1): ¹H-NMR (DMSO-d₆, 500 MHz) δ: 8.32 (1H, s, H-2), 7.69 (1H, d, J=8.5 Hz, H-5), 7.36 (2H, d, J=8.5 Hz, H-2′, 6′), 6.99 (1H, d, J=8.5 Hz, H-6), 6.80 (2H, d, J=8.5 Hz, H-3′, 5′), 3.85 (3H, s, OCH₃); ¹³C-NMR (DMSO-d₆, 125 MHz) δ: 175.2 (C-4, C═O), 157.4 (C-4′), 155.8 (C-7), 153.0 (C-2), 151.0 (C-9), 135.0 (C-8), 130.4 (C-2′, 6′), 123.5 (C-3), 122.7 (C-1′), 121.0 (C-5), 117.2 (C-10), 115.8 (C-6), 115.3 (C-3′, 5′), 61.0 (OCH₃). The HMBC spectrum revealed a methoxyl proton signal at δ 3.85(s) correlated to carbon resonance at δ 135.0 (C-8). Based on these spectral data and with the comparison of ¹H-NMR and ¹³C-NMR data in the literature [Chin. Pharm. J. 2012; 47:179-18], compound (1) was characterized as 7,4′-dihydroxy-8-methoxy-isoflavone, as shown in FIG. 5.

Compound (2) was obtained as pale yellow powder, showed an [M-H]⁺ ion peak at m/z: 283, and ¹H-NMR (DMSO-d₆, 500 MHz) δ: 8.33 (1H, s, H-2), 7.57 (1H, d, J=8.8 Hz, H-5), 7.37 (2H, d, J=8.5 Hz, H-2′, 6′), 7.20 (1H, d, J=8.8 Hz, H-6), 6.80 (2H, d, J=8.5 Hz, H-3′, 5′), 3.91 (3H, s, OCH₃); ¹³C-NMR (DMSO-d₆, 125 MHz) δ: 175.7 (C-4, C═O), 157.3 (C-4′), 153.4 (C-2), 151.4 (C-7), 146.1 (C-9), 134.9 (C-8), 130.4 (C-2′, 6′), 123.2 (C-3), 122.8 (C-1′), 118.7 (C-10), 115.4 (C-5), 115.3 (C-3′, 5′), 110.5 (C-6), 56.6 (OCH₃). The methoxyl group of the compound was demonstrated at the C-7 position of the isoflavone by ¹H-¹³C long-range correlation between H of OCH₃ and C-7 of isoflavone. By comparing these data with the values in the literature [Chin. Pharm. J. 2012; 47:179-181], compound (2) was identified as 8,4′-dihydroxy-7-methoxy-isoflavone, as shown in FIG. 5.

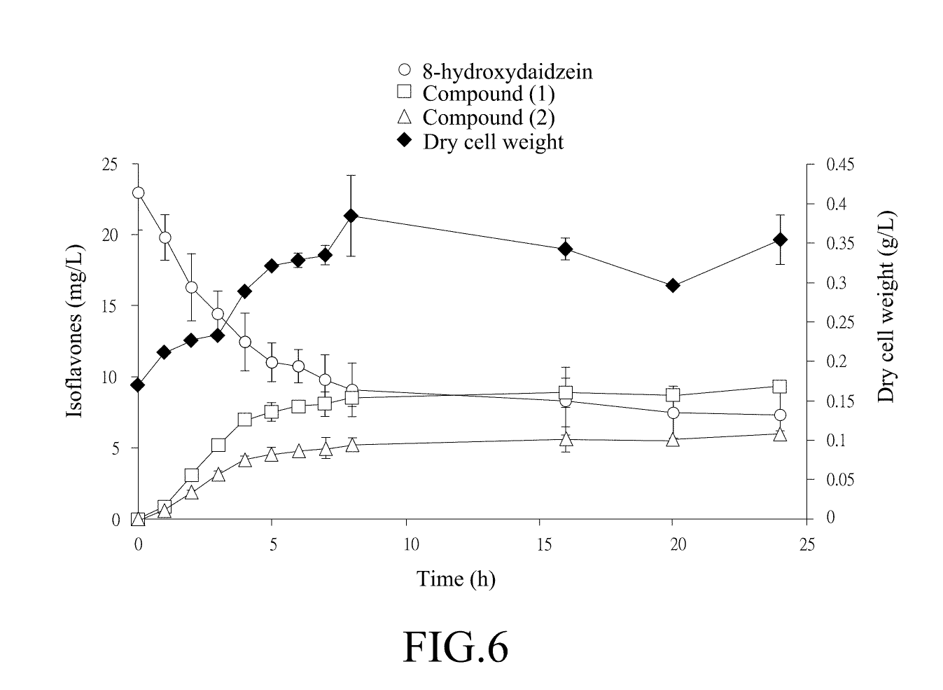

For the further scale-up fermentation, a seed culture of recombinant E. coli (100 mL) was inoculated into a 5 L fermenter containing 2.5 L LR medium supplemented with 0.4% glycerol, followed by cultivation with aeration (0.5 vvm) and agitation (280 rpm) at 37° C. The fermentation broths under different IPTG induction treatments were collected, condensed under a vacuum, and then crystallized by freeze-drying. Referring to FIG. 6, production yields of methoxy-isoflavones according to the present invention are revealed. The results are shown while the Y axis represents isoflavones profiles (◯, □, Δ) and dry cell weight (♦), and the X axis includes different treatment conditions. The maximum production yields of compounds (1) and (2) in the biotransformation were 9.3 and 6.0 mg/L, respectively. In the results, the products accumulated rapidly within the initial 6 h after induction and then at a very slow rate.

### Experiment 3: Determination of Cell Viability and Melanin Content

The ranges of non-toxic concentrations of the compounds toward the B16 cells was first determined with the 3-(4,5-dimethylthiazol-2-yl)-2,5-diphenyltetrazolium bromide (MTT) method. Mouse B16 melanoma cells (4A5) was purchased from the Bioresources Collection and Research Center (BCRC, Food Industry Research and Development Institute, Hsinchu, Taiwan). Determinations of cell viability and melanin content were performed as previously reported [Appl. Microbiol. Biotechnol. 2007; 73:1169-1172] and described briefly below. Dulbecco's modified Eagle's medium (DMEM) containing 10% (v/v) fetal bovine serum was used to cultivate mouse B16 cells, which were incubated at 37° C. in a humidified, CO₂-controlled (5%) incubator. After 1 day of incubation, the cells were treated with tested drugs in the presence of a melanogenesis stimulation agent (400 μM of 3-isobutyl-1-methylxanthine (IBMX)) for another 2 days. Then, the drug-treated cells were harvested and the cell viability and melanin content were measured according to the previous study [Curr. Pharma. Biotechnol. 2015; 16:1085-1093]. In this experiment, cells only treated with IBXM is used as a negative control and cells treated with 20 μM danazol is used as a positive control.

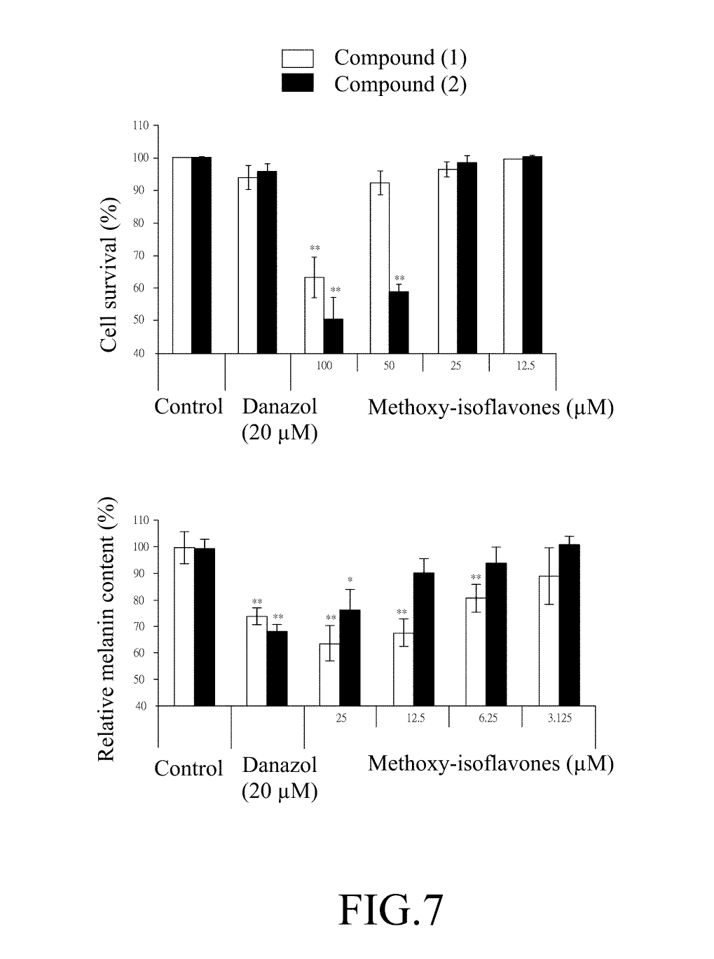

Referring to FIG. 7, cytotoxicity of methoxy-isoflavones and effect of methoxy-isoflavones under different treatment conditions on melanogenesis according to the present invention are revealed. The X axis includes different treatment conditions, and the Y axis of the results represents cell survival rate or relative melanin content. Both of the cell survival rate and the relative melanin content are expressed as percentage of control (% of control). The results shows that both compound (1) (open bars) and compound (2) (filled bars) did not exhibit cytotoxicity under a concentration of 25 μM. Accordingly, the 25 μM was used as the maximum concentration in the determination of the inhibition of melanogenesis.

The result in FIG. 7 also showed that both compound (1) and compound (2) dose-dependently inhibited melanogenesis of B16 cells in non-toxic concentrations. Specifically, compared with the control group, compound (1) with concentration of 6.25 μM and 25 μM show inhibition of melanogenesis in B16 cells, especially the melanin content of the group treated with 25 μM compound (1) is decreased to 63.5%. Moreover, the inhibition by compound (1) was greater than that produced by danazol, which has previously been identified as a potent melanogenesis inhibitor [Arch. Pharm. Res. 2010; 33:1959-19].

In summary, methoxy-isoflavones manufactured by the present method do have inhibitory effect on melanogenesis. Thereby methoxy-isoflavones can be used as material for whitening cosmetics such as water base cosmetics, emulsion cosmetics, ointment base cosmetics, power cosmetics, etc. The cosmetic composition further includes at least one commonly-used cosmetically acceptable adjuvant selected from solvents, gelling agents, active agents, preservatives, antioxidants, screening agent, chelating agents, surfactants, thickening agent, perfumes, and odor absorbers. The cosmetic composition can also be used together with at least one external use agent selected from whitening agents, humectants, anti-inflammatory agents, ultraviolet absorbers, plant extracts, anti-acne agents, antipsoriatic agents, antiagers, antiwrinkle agents and wound-healing agents.

Compared with the technique available now, the present invention has the following advantages:

1. The present invention has proven that 8-hydroxydaidzeins can be converted to methoxy-isoflavones by biotransformation for the first time, and the production profile of the two methoxy-isoflavones, 7,4′-dihydroxy-8-methoxy-isoflavone and 8,4′-dihydroxy-7-methoxy-isoflavone, can be as high as 9.3 and 6.0 mg/L, respectively.
2. The present invention has proven that non-toxic concentrations of methoxy-isoflavones, especially 7,4′-dihydroxy-8-methoxy-isoflavone, has an inhibitory effect on melanogenesis.

What is claimed is:

1. A method of manufacturing methoxy-isoflavones by biotransformation comprising the steps of:
(a) synthesizing a nucleic acid sequence having a SpOMT2884 gene of Streptomyces peucetius, a first restriction enzyme cleavage site and a second restriction enzyme cleavage site, wherein the first and the second restriction enzyme cleavage sites are respectively located upstream and downstream of the SpOMT2884 gene of Streptomyces peucetius;
(b) cleaving the nucleic acid sequence with the first restriction enzyme and the second restriction enzyme;
(c) cleaving an expression vector with the first restriction enzyme and the second restriction enzyme, and cloning the nucleic acid sequence into the expression vector to form a cyclic recombinant plasmid; and
(d) transforming the cyclic recombinant plasmid into a microbial expression system and incubating the microbial expression system in a medium containing 8-hydroxydaidzeins therein for generating methoxy-isoflavones.

2. The method of manufacturing methoxy-isoflavones by biotransformation as claimed in claim 1, wherein the SpOMT2884 gene of Streptomyces peucetius encodes an O-methyltransferase and comprises an amino acid sequence of SEQ ID NO:1.

3. The method of manufacturing methoxy-isoflavones by biotransformation as claimed in claim 1, wherein the first restriction enzyme is NdeI and the second restriction enzyme is XhoI.

4. The method of manufacturing methoxy-isoflavones by biotransformation as claimed in claim 1, wherein the expression vector is pETDuet™.

5. The method of manufacturing methoxy-isoflavones by biotransformation as claimed in claim 1, wherein the microbial expression system is Escherichia coli.

6. The method of manufacturing methoxy-isoflavones by biotransformation as claimed in claim 1, wherein the methoxy-isoflavones are 7,4′-dihydroxy-8-methoxy-isoflavone and 8,4′-dihydroxy-7-methoxy-isoflavone.

7. The method of manufacturing methoxy-isoflavones by biotransformation as claimed in claim 6, wherein the methoxy-isoflavones are used for inhibiting melanogenesis by applying an effective dose of the methoxy-isoflavones to a subject in need, and wherein the effective dose is 6.25 μM-25 μM.

8. The method of manufacturing methoxy-isoflavones by biotransformation as claimed in claim 7, wherein the methoxy-isoflavones are further used in a cosmetic composition for whitening.
